(19) United States
(12) Patent Application Publication
(10) Pub. No.: US 2022/0225754 A1
(43) Pub. Date: Jul. 21, 2022

(54) TOOTHBRUSH

(71) Applicant: ORTHONU, LLC, RUMSON, NJ (US)

(72) Inventors: Sima Yakoby Epstein, Rumson, NJ (US); Douglas Freeman Melville, JR., Simsbury, CT (US)

(21) Appl. No.: 17/498,646

(22) Filed: Oct. 11, 2021

Related U.S. Application Data
(60) Provisional application No. 63/140,247, filed on Jan. 21, 2021.

(51) Int. Cl. A46B 9/04; A46B 5/00; A46D 1/00

(52) U.S. Cl. CPC A46B 9/04; A46B 2200/1066; A46D 1/0207; A46B 5/00

(57) ABSTRACT

A toothbrush for brushing a user's teeth is a T-shaped member having a handle forming a lower part of the T and a head forming the upper, transverse part of the T. A plurality of bristles extends outwardly from a surface of the head. A combination of at least the head and the plurality of bristles together provides about a 45-degree brushing angle relative to a side surface of a tooth of a user.

### CROSS-REFERENCED TO RELATED APPLICATION

This application claims priority to U.S. Provisional Application No. 63/140,247, filed Jan. 21, 2021, the disclosure of which is incorporated herein by reference in its entirety.

### FIELD

The present invention concerns a toothbrush. In particular, the present invention concerns a toothbrush that is particularly useful with braces and orthodontic treatments.

### BACKGROUND

In the orthodontic care of patients, there are at least three areas of concern that must be addressed, and which are currently under-addressed in consumer solution products. These include 1) emergency care, 2) oral hygiene, and 3) visual challenges. Emergency care is needed to address discomfort, pain, sensitivity, ulcerations, dry mouth, and the like.

Oral hygiene involves long term oral care and prevention. Oral hygiene includes maintenance care for preventing halitosis and preventing or treating gingivitis and the increase in gram negative bacteria on the tongue and periodontium that are associated with orthodontia care. Avoiding gingivitis and reducing gram negative bacteria in a patient's mouth can prevent and/or deter bone loss and inflammation. This can help to maintain a healthy mouth, tongue, gums, and fresh breath.

Visual challenges include the appearance of white decalcifications on teeth due to the start of cavities that can be attributed to an increase of gram-positive bacteria. Other visible challenges include broken brackets, tooth and elastic discoloration, food stuck between teeth, and staining of enamel.

Gram negative bacteria lives on the tongue and inside the gum line around the periodontium. Gram negative bacteria is a main cause of halitosis in the mouth. Gram positive bacteria is found in the plaque accumulation of the pellicle that forms on the enamel of the tooth that then starts the decalcification process that can lead to cavities, also causing halitosis. Targeting these two types of bacteria with different ingredients and tools and differentiating them can solve many issues inherent in the changes in microbiome during orthodontic treatment.

Patients undergoing orthodontic treatment often undergo challenges. Shifting teeth can trigger increases in bacterial loads. Because of the lack of home care solutions, patients often simply live with the bad breath and plaque that builds up on their teeth. In particular, on-the-go solutions and home-care solutions are needed.

It is especially important for patients to brush their teeth during orthodontic treatment because food gets stuck between the brackets/bands and the patient's teeth. Food and bacteria left untreated on your teeth can cause many issues, from short term bad breath to longer term cavities and gingivitis. However, its cumbersome to brush or floss effectively because user's find it hard to manipulate around different bracket shapes, sizes, and excess flash composite around clear aligner attachments. Brushing teeth with brackets or aligner attachments is technique sensitive to remove built up bacteria and plaque around the bracket/elastic attachment complex. Furthermore, due to an increase of bacteria and inflammation of gums in patients with braces, patient's often experience sensitivity, bleeding and ultimately a reluctance to brush around highly susceptible areas.

One currently recommended technique for teeth brushing is the Modified Bass Technique, which is well known and involves angling a toothbrush at a 45-degree angle to your gum line. An illustration of the Modified Bass Technique is shown in FIG. 1. Brushing in either a back-and-forth motion or a circular motion occurs with firm, yet gentle pressure 15 to 20 times before moving to the next area. All teeth are brushed on the outer surface and then the backsides of the teeth are cleaned using the same motions. For the front teeth, the toothbrush is held in a vertical position behind the front teeth and brushed up and down, using the bristles on the toe of the toothbrush. Lastly, the chewing surface of the molars and the tongue are brushed. Each tooth is brushed well with approximately 20 repeated strokes and then, when finished, the toothbrush is flicked down the tooth, away from the gum line. It is recommended that brushing occur for 2 minutes, at least twice per day. The purpose behind angling the brush at a 45-degree angle to the gums is to scrub off plaque, commonly known as biofilm, from underneath the gums before it can cause gingivitis.

Another brushing technique is Stillman's Brushing Technique, illustrated in FIG. 2. With this technique, the goal is to remove plaque from above the gum line, which is beneficial for patients with gingivitis. In this technique, the patient angles the toothbrush, so the bristles are positioned at a 45-degree angle toward the gum line. The bristles should be half on the gums and half on the tooth's surface. The patient then makes approximately 20 short, light horizontal strokes, which results in plaque being removed from above the gum line. The movement also helps to stimulate the gums. Once the patient has finished with a set of teeth, they then move to the next set of teeth and repeat.

Yet another technique for brushing teeth is Charter's Brushing Technique, which is illustrated in FIG. 3. This technique is targeted toward patients who have open inter dental spaces, those wearing fixed partial dentures, or those with braces. In this technique, the patient positions the toothbrush so that the bristles are at a 45-degree angle and the patient directs them coronally with mild vibratory strokes to remove plaque from teeth, brackets, and arch wire. Then the patient changes direction so the bristles remove plaque from teeth below the brackets and arch wire.

### SUMMARY

The invention concerns a toothbrush, as depicted and claimed. The toothbrush is designed to aid a wearer of braces or aligners in improving their mouth hygiene.

### BRIEF DESCRIPTION OF THE DRAWINGS

FIG. 1 depicts an illustration of a prior art tooth brushing technique;

FIG. 2 depicts an illustration of another prior art tooth brushing technique;

FIG. 3 depicts an illustration of yet another prior art tooth brushing technique;

FIG. 4 depicts a right-side perspective view of an example toothbrush according to the invention;

FIG. 5 depicts a left side perspective view of the toothbrush of FIG. 4;

FIG. 6 depicts a rear view of the toothbrush of FIG. 4;

FIG. 7 depicts a top view of the toothbrush of FIG. 4;

FIG. 8 depicts a bottom view of the toothbrush of FIG. 4;

FIG. 9 depicts a left side view of the toothbrush of FIG. 4;

FIG. 10 depicts a rear view of the toothbrush of FIG. 4;

FIG. 11 depicts a right-side cross-sectional view of the toothbrush of FIG. 4;

FIG. 12 depicts a right-side perspective view of an alternative example toothbrush according to the invention;

FIG. 13 depicts a left side perspective view of the toothbrush of FIG. 12;

FIG. 14 depicts a rear view of the toothbrush of FIG. 12;

FIG. 15 depicts a top view of the toothbrush of FIG. 12;

FIG. 16 depicts a bottom view of the toothbrush of FIG. 12;

FIG. 17 depicts a left side view of the toothbrush of FIG. 12;

FIG. 18 depicts a rear view of the brush of FIG. 12;

FIG. 19 depicts a right-side cross-sectional view of the toothbrush of FIG. 12;

FIG. 20 depicts a rear view of an alternative embodiment of a toothbrush according to the invention;

FIG. 21 depicts a right-side view of an alternative embodiment of a toothbrush according to the invention;

FIG. 22 depicts a rear view of yet another alternative embodiment of the toothbrush according to the invention;

FIG. 23 illustrates use of the toothbrush according to the invention in brushing teeth;

FIG. 24 depicts a rear view of another alternative embodiment of the toothbrush according to the invention, with the toothbrush having a head portion that differs from prior embodiments;

FIG. 25 depicts a front view of the toothbrush of FIG. 24; and

FIG. 26 depicts a side view of the toothbrush of FIG. 24 showing the shape of the handle.

### DETAILED DESCRIPTION

The present invention provides a toothbrush 10 that is designed for use with braces and/or aligners that are used for straightening teeth. As discussed above, hygiene of the mouth is hindered when patients undergo orthodontia treatment. The toothbrush 10 is designed to improve mouth hygiene.

There are many different patents that describe a toothbrush head that is angled at a 45° angle so that a user can properly brush their teeth using the Modified Bass, Stillman, Charter, or other methods of brushing. However, these patents fail to consider that a user does not hold their toothbrush completely vertical during brushing. These patents also fail to consider the shape of the handle of the brush and to take this into consideration when calculating an angle for the bristles. These prior methods also fail to consider that the user who uses a vertical toothbrush will incline the brush at an angle to get the brush into their mouth under the cheek or lips, and that the user uses their hand/arm to do the brushing, which is also not mechanically aligned with the mouth. The present invention takes these factors into account and has determined that the user typically brushes their teeth at an angle. With the toothbrush disclosed herein, which is a vertical toothbrush, the angle that a typical user will angle the brush is approximately 20 degrees.

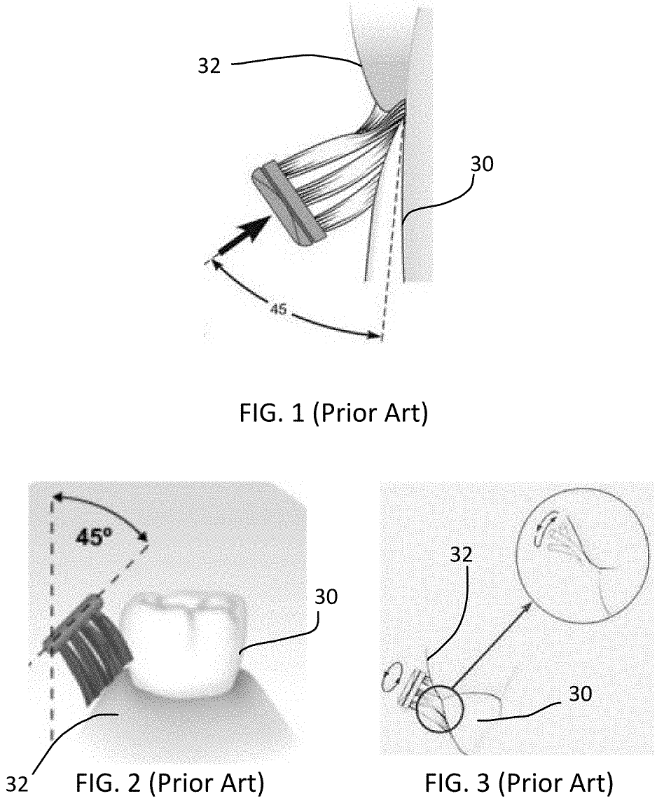

According to the invention, the specially designed orthodontic toothbrush 10 described herein is T-shaped, with a curved handle 12 forming the lower part of the T-shape that is positioned substantially vertically during brushing, and an upper part that forms the upper end of the T-shape is a head 14 that extends transversely, or at an approximately 90-degree angle relative to the longitudinal axis Y-Y of the handle 12 (with the longitudinal axis Y-Y extending along the length of the handle 12). Bristles 16 are coupled to the head 14 and extend upwardly therefrom to provide a brushing angle 4 of 45° during use. In contrast, conventional non-vertical toothbrushes have a handle and a toothbrush head that it is positioned parallel to the teeth and bristles that are at a 90-degree angle relative to the handle, as shown in FIGS. 1-3. The toothbrush 10 described herein helps to optimize brushing and hygiene because it reaches between brackets of braces and hard to reach places, including between the gums and teeth.

Several different designs for toothbrushes 10 are shown below, with one brush having a shorter handle 12 and others having a longer, thinner handle 12. The shorter, thicker toothbrush may be easier to use, but both brushes 10 have the same benefits of the angled bristles 16 and substantially vertical brushing. Moreover, the bristles 16 are angled upwardly at an angle. This may be accomplished by angling the bristles 16 relative to the upper surface 18 of the head 14, or by angling the upper surface 18 of the head 14 so that the bristles 16 extend perpendicular to the upper surface 18. Because the upper surface 18 of the head 14 is angled, the bristles 16 are angled in the same manner as the angle of the head 14.

The toothbrush 10 is configured for use vertically instead of horizontally. The toothbrush 10 has angled bristles 16 that are designed to easily permit a patient to brush the correct way above and below the brackets and attachments of braces, and around the sensitive gumline to optimize the cleaning process. The toothbrush 10 substantially removes patient error in the brushing process and design of the toothbrush 10 aids in the cleaning process.

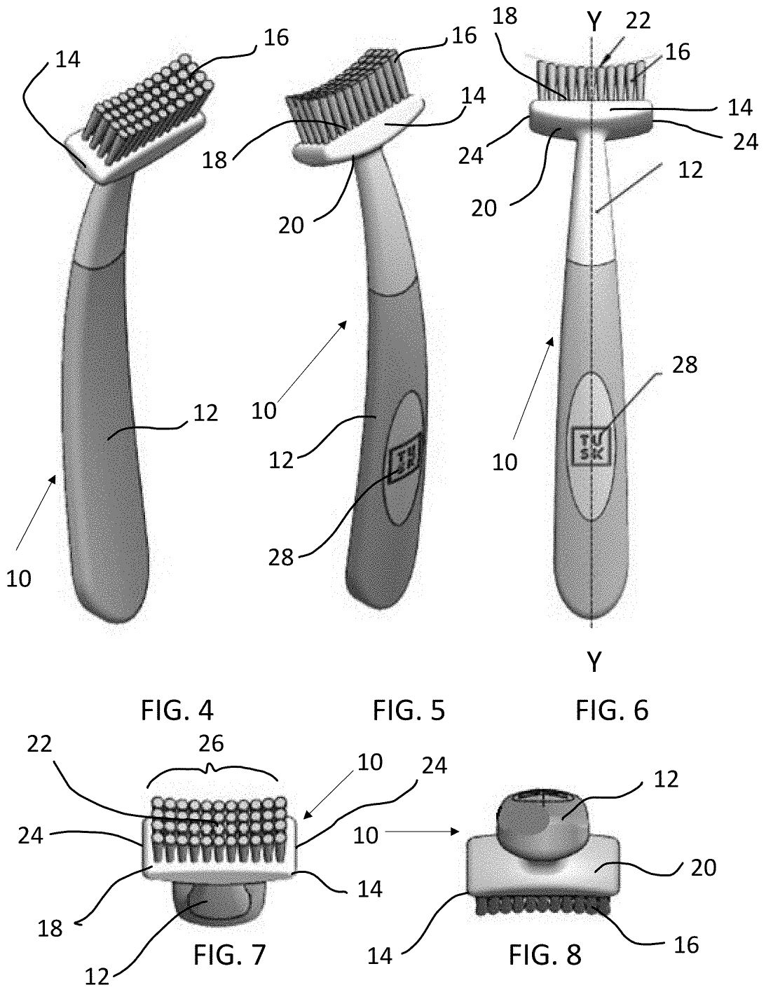

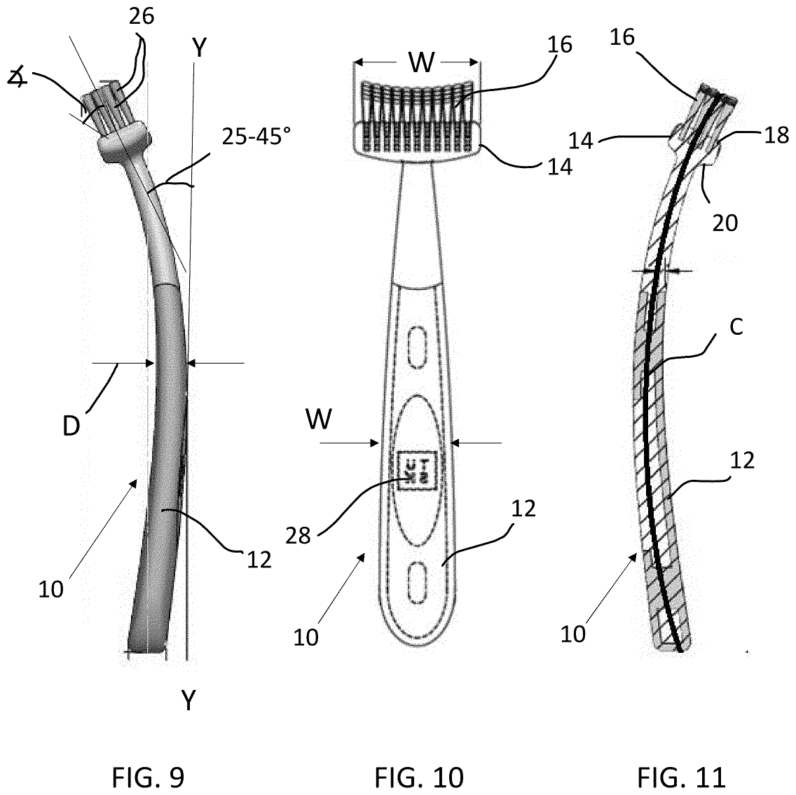

FIGS. 4-11 depict a first embodiment of the toothbrush 10 according to the invention. In this embodiment, the toothbrush 10 is T-shaped. It has a handle 12 at the bottom and a head 14 at the top. The head 14 forms the crossbar of the T-shape, and the handle 12 forms the lower part of the T-shape. The handle 12 is arc-shaped or curved. It may be a continuously curved shape at a constant radius or may have multiple radiuses of curvature. A longitudinal axis Y-Y is defined along the length of the toothbrush 10, as shown in FIGS. 6 and 9.

The head 14 has an upper surface 18 that is substantially flat. The upper surface 18 of the head 14 could alternatively have a shape that is not flat, such as curved, stepped, or angled, among other shapes. The lower surface 20 of the head 14 is curve-shaped, such that the center 22 of the head 14 is thicker than the ends 24 of the head 14. This provides stability to the head 14 and support for the bristles 16. The head 14 has sufficient thickness and strength to permit bristles 16 to be inserted into the upper surface 18 of the head 14.

As is evident, the head 14 of the present toothbrush 10 is different from a traditional toothbrush 10, which has bristles 16 that extend transversely from the handle 12. In this case, the head 14 extends transversely from the handle 12 and the bristles 16 extend outwardly from an upper surface 18 of the head 14. The bristles 16 are shown grouped in bunches 26 that extend substantially perpendicular to the upper surface 18 of the head 14. The head 14 is angled at an angle 4 relative to the longitudinal axis Y-Y because of its connection to the upper end of the handle 12, which is angled relative to the vertical. Alternatively, the head 14 itself may be angled to provide a desired angle for brushing. The bristles 16 are shown being aligned with the angle 4 of the head 14 but could be at an angle 4 that is different from the angle 4 of the head 14. For example, the head 14 could be angled at a first angle and the bristles 16 could be angled at a second angle to provide the user with a brushing angle of about 45°.

As shown in the rear view in FIGS. 6 and 10, the handle 12 of the toothbrush 10 is tapered and has a narrower width W at the upper end and transitions from a narrow width W at an upper end to a wider width W at a lower end. The handle 12 is also rounded at the lower end to provide an appealing shape and no sharp edges.

As shown in FIGS. 9 and 11—the side views, the handle 12 has a narrower depth D along its length than the width W. The depth D is narrower at the upper end of the handle 12 than at the lower end of the handle 12. The depth D, as shown, is always less than the corresponding width W of the handle 12 along the length thereof, although this is not required in all cases. FIG. 9 also depicts how the upper end of the handle 12 may extend at an angle 4. This angle 4 may alternatively be 45°, 40°, 35°, 30°, 25°, or 20°, among other angles. When a brushing angle is taken into consideration, the angle of the upper end of the handle along with the angle of the head is typically 25°, but could be other angles, such as 20°, or 15°.

The bristles 16 are shown extending outwardly from the head 14 and aligning with the angle 4 of the upper end of the handle 12. Alternatively, the upper surface 18 of the head 14 could be angled differently than shown to assist in providing a brushing angle of 45°. Alternatively, the bristles 16 could be angled while the upper surface 18 of the head 14 of the toothbrush 10 remains as shown. It is preferred that the toothbrush 10 provide an angle of 45° when brushing. As noted, this can be provided by a combination of the angle of the toothbrush 10 as held by a user, which is typically at an angle of approximately 20° from vertical, the angle of the bristles 16 and the angle of the handle 12. In one embodiment, the brushing angle is 20° from the vertical and the bristle angle is 25° to provide a total angle of 45°.

The toothbrush 10 is substantially symmetrical from side to side but is not symmetrical from front to rear. The toothbrush 10 may be made of multiple parts that are adhered or fitted together in any known way. Four rows of bristle bunches 26 are shown disposed on the head 14 of the toothbrush 10, with eleven columns to form a rectangular shape for the bristles 16. Other shapes and sizes for the bristles 16 may be used. The bristles 16 may be all the same length or different lengths.

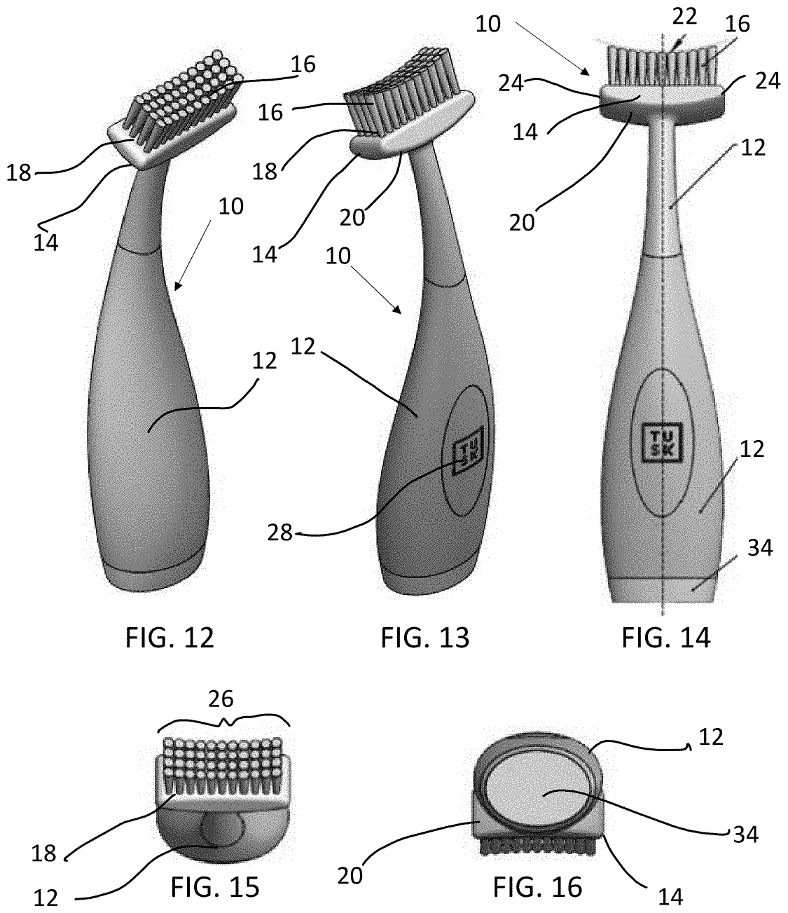

FIGS. 12-19 depict another embodiment of the toothbrush 10 where the handle 12 has a different shape than in the embodiment shown in FIGS. 4-11. In particular, the depth D of the handle 12 is more bulbous in the lower part of the handle 12 and narrower in depth D at the upper part of the handle 12. The head 14 of the toothbrush 10 is substantially the same as described above in FIGS. 4-11. The width W of the handle 12 is at its smallest width dimension where it attaches to the lower surface of the head 14 and tapers outwardly as it extends downwardly to a maximum width W toward a bottom end of the handle 12. The handle 12 narrows slightly below the widest part of the handle 12 at the bottom end thereof and has a substantially flat bottom end.

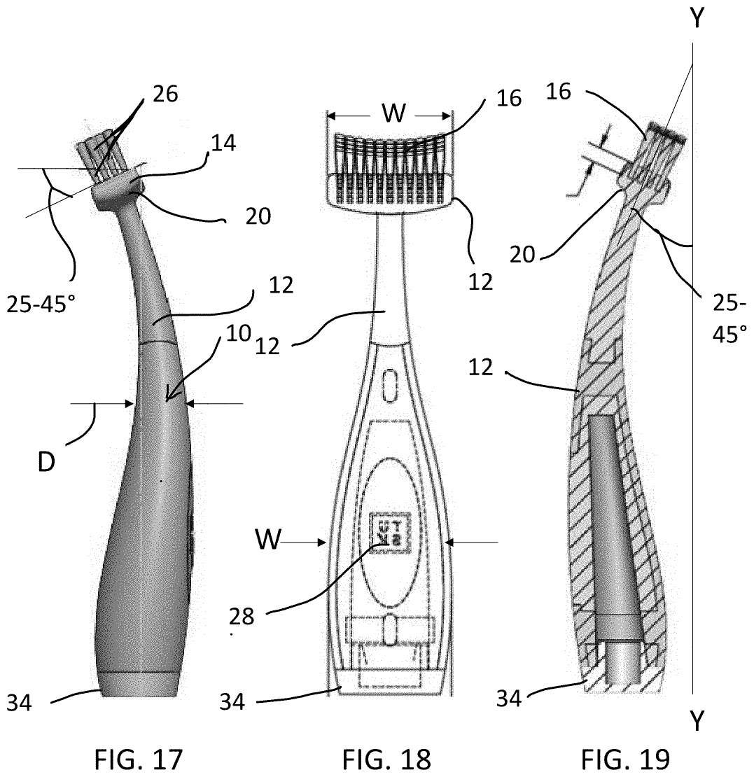

As shown best in FIGS. 17 and 19, the handle 12 has a depth D that is generally less than the width W of the toothbrush 10. The upper end of the handle 12 is narrower than the lower end of the handle 12, which is more bulbous in shape. As shown in FIG. 18, the toothbrush 10 includes an area 28 where advertising material, a logo, indicia, instructions, or otherwise, may be positioned on the handle 12.

FIG. 19 depicts a longitudinal axis Y-Y extending along the length of the toothbrush 10 and shows that the bristles 16 are angled at approximately an angle, which may be 25°, or another angle, to provide a brushing angle of 45°. FIG. 17 depicts how the upper surface 18 of the head 14 is also angled at an approximate 25° angle. The head 14 and/or bristles 16 can be angled at a different angle depending on the typical angle of brushing associated with the toothbrush 10, which is based in part upon the shape of the handle 12.

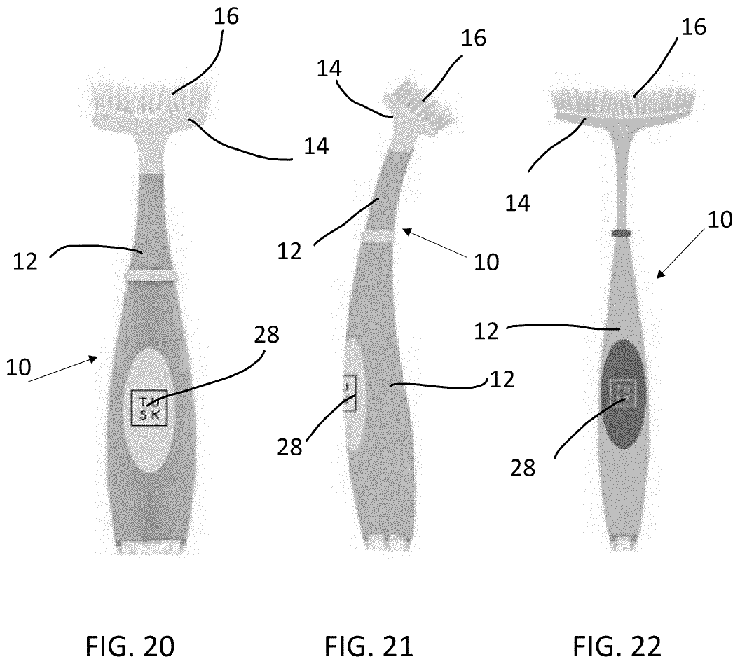

FIGS. 20 and 21 represent an alternative embodiment of the toothbrush 10 where the upper surface 18 of the head 14 is curved, or arc-shaped, with the curve having a substantially constant radius. As shown, the center 22 of the head 14 is recessed slightly relative to the ends 24 of the head 14. This curved shape can be designed to mimic the curvature of rows of teeth in the mouth. Like with the embodiment shown in FIGS. 12-19, the handle 12 has a more bulbous shape.

FIG. 22 depicts another embodiment where the handle 12 is narrower than that shown in FIG. 20, but with a head 14 that is wider than the head 14 shown in prior embodiments. Like FIGS. 20-21, the upper surface 18 of the head 14 is curved instead of flat.

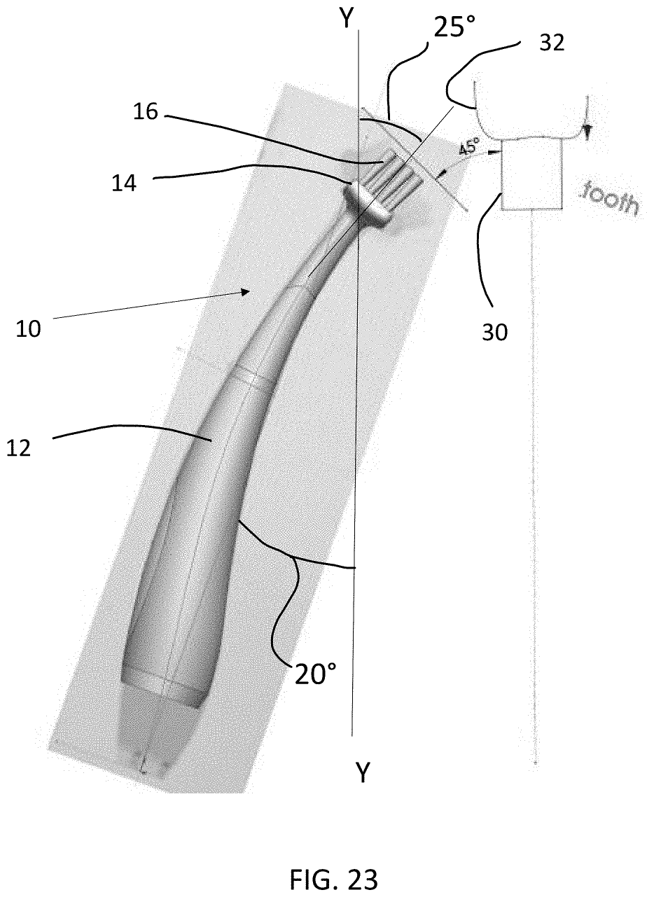

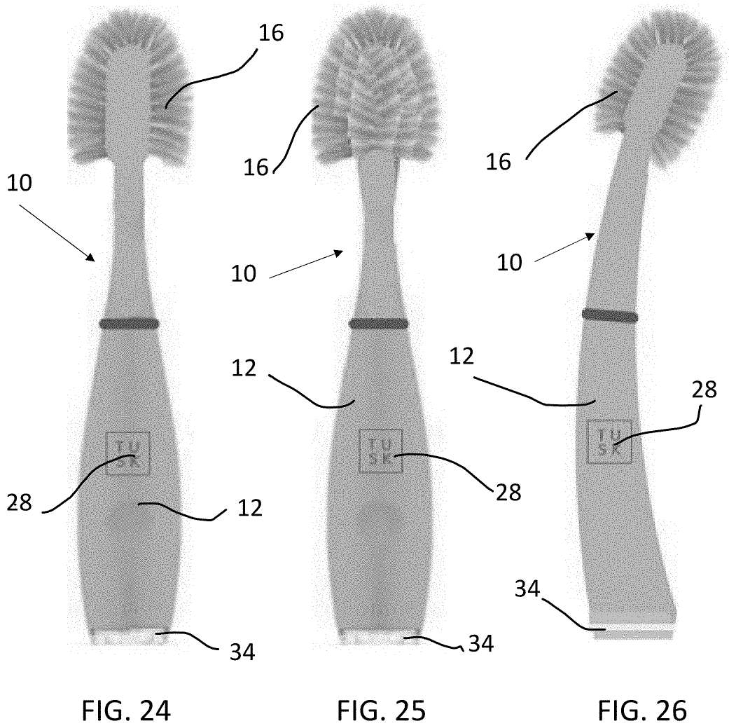

FIG. 23 depicts a view of the toothbrush 10 adjacent a user's tooth 30. In use, the patient holds the handle 12 in a substantially vertical position that is slightly angled, and the user rubs the teeth 30 up and down from the gums 32 to the bottom of the teeth 30. A typical angle for a user to hold a toothbrush 10 relative to their teeth 30 is 20° relative to vertical. A group of teeth 30 is brushed at a single time, with the number of teeth 30 being covered dependent upon the width W of the head 14 of the toothbrush 10. The patient can use an up and down motion or a circular motion. Once one section of teeth 30 is completed, the patient then moves on to another section of teeth 30. To brush the upper teeth 30, the brush head 14 is positioned above the handle 12 and, to brush the lower teeth 30, the brush head 14 is positioned below the handle 12. The angle of the toothbrush 10 permits the user to put the toothbrush 10 in their mouth while still being able to maneuver the toothbrush 10 to brush the teeth 30. As shown the angle that the user holds the toothbrush is approximately 20° and the angle of the brush head and bristles is about 25°.

Other ways to optimize brushing and hygiene of the teeth 30 is to provide specialized paste, gels or treatment fluids that are designed to remove enamel stains, eliminate plaque buildup, and reduce the white decalcifications on the enamel that can lead to cavities. The patient may use a toothpaste or toothpaste gel to brush the teeth 30 and brackets and a separate gel for treating the gums 32.

The toothbrush 10 can include a detachable handle 12 that provides different paste or treatment fluids to dispense to the teeth 30, or a UV-C blue or red light (not shown) can emanate from the toothbrush 10 and be applied to teeth to reduce the buildup of bacteria on the teeth 30 and around the gum line 32. A special orthodontic paste may include baking soda, hydroxyapatite, xylitol, and peppermint oil. Other ingredients and/or different ingredients may alternatively be used. The orthodontic toothbrush 10 may be offered as part of a kit and/or system for better oral health during treatment with braces as well as for regular brushing. The toothbrush 10 may include areas 28 on the surface thereof for applying logos, indicia, or other markings.

The toothbrush 10 can be manual or electric/battery powered.

The toothbrush 10 can be made of any known materials, including plastics, wood, and the like. The bristles 16 can be natural, such as Miswak bristles, or synthetic, such as nylon. Other materials may also be used, including other synthetic and natural materials. Miswak is a traditional chewing stick prepared from roots, twigs, and stem of the Salvadora persica plant. Chewing sticks of this nature have been used in many parts of the world for thousands of years. Miswak possesses antibacterial, anti-fungal, anti-viral, anti-carcinogenic, and anti-plaque properties. The present invention proposes using miswak fibers that are embedded into the head 14 of the toothbrush 10 and that serve as bristles 16. Alternatively, synthetic bristles, such as nylon, can be used, among other known types of materials. The bristles 16 can alternatively be Miswak extract infused nylon bristles.

The toothbrush 10 can have increased efficacy by utilizing a removable handle 12 with a pouch of specialized paste or treatment fluids to dispense to the teeth that are designed to remove enamel stains, eliminate plaque buildup, and reduce the white decalcifications on the enamel that lead to cavities. A special orthodontic paste may include baking soda, hydroxyapatite, xylitol, and peppermint oil. Other ingredients and/or different ingredients may alternatively be used. The orthodontic toothbrush 10 may be offered as part of a kit and/or system for better oral health during treatment with braces as well as for regular brushing.

A toothbrush 10 for brushing a user's teeth is a T-shaped member having a handle 12 forming a lower part of the T and a head 14 forming the upper, transverse part of the T. A plurality of bristles 16 extends outwardly from a surface of the head 14. A combination of at least the head 14 and the plurality of bristles 16 together with an angle at which the toothbrush is held provides about a 45-degree brushing angle relative to a side surface of a tooth 30 of a user.

A normal brushing position of a user may provide an angle of about 20° relative to a side surface of a tooth 30 of a user (e.g., the vertical) and the combination of the head 14 and the plurality of bristles 16 provides an angle of about 25° to provide an overall brushing angle of about 45°. Other brushing angles and bristle angles may be used depending on the configuration of the toothbrush 10.

The handle 12 may be curved at a substantially constant radius curve from a top end to a bottom end. The head 14 may have an upper surface 18 and a lower surface 20, and the upper surface may be substantially flat 18. A bottom end of the handle 12 may be substantially flat to permit the toothbrush 10 to stand upright on a surface. The handle 12 may be curved from front to rear. The handle 12 may be wider at a bottom end thereof than at a top end where it couples with the head 14.

The plurality of bristles 16 may be grouped into bunches 26 and each of the bunches 26 may extend at substantially the same angle as the other bunches 26 of bristles 16. The plurality of bristles 16 within each bunch 26 may extend in the same direction and some of the bristles 16 are perpendicular to the upper surface 18 of the head 14. The plurality of bristles 16 may be made of miswak, nylon, or miswak infused nylon. The bristles 16 may be angled at an angle of 45° relative to a side surface of a user's tooth.

The head 14 may have a substantially flat upper surface 18 and an arc-shaped lower surface 20, with the bristles 16 extending from the upper surface 18. The head 14 may have a width W that is greater than the width W of the handle 12. An upper part of the handle 12 may be angled, and the plurality of bristles 16 may align with the angle of the upper part of the handle 12. The handle 12 may have a width W that is greater than a depth D thereof.

In another embodiment, the toothbrush 10 includes a T-shaped member having a handle 12 forming the lower part of the T and a head 14 forming the transverse part of the T. A plurality of bristles 16 extends outwardly from the head 14. The combination of the head 14, the bristles 16, and a normal brushing position of a user together provides a 45-degree angle relative to a side surface of a tooth for brushing.

The normal brushing position of a user may provide an angle of about 20° relative to a side surface of a user's tooth. The handle 12 of the brush may be angled at an upper end thereof at an angle of about 25° relative to the side surface of a tooth of a user to provide a total angle of 45°.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. All percentages and averages are by weight unless the context indicates otherwise. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A toothbrush for brushing a user's teeth comprising:
a T-shaped member having a handle forming a lower part of the T and a head forming the upper, transverse part of the T; and
a plurality of bristles extending outwardly from an upper surface of the head;
wherein a combination of at least the head and the plurality of bristles together provides about an overall 45° brushing angle relative to a side surface of a tooth of a user.

2. The toothbrush of claim 1, wherein a typical brushing angle of a user provides an angle of about 20° relative to a side surface of a tooth of a user and the combination of the head and the plurality of bristles provides an angle of about 25° to provide an overall brushing angle of about 45°.

3. The toothbrush of claim 1, wherein the handle is curved at a substantially constant radius curve from a top end to a bottom end.

4. The toothbrush of claim 1, wherein the head has an upper surface and a lower surface, and the upper surface is substantially flat.

5. The toothbrush of claim 1, wherein the plurality of bristles is grouped into bunches and each of said bunches extends at substantially the same angle as the other bunches of bristles.

6. The toothbrush of claim 5, wherein the head has a thickness sufficient to hold the bunches of bristles.

7. The toothbrush of claim 6, wherein the plurality of bristles within each bunch extends in the same direction and at least some of said bristles are perpendicular to the upper surface of the head.

8. The toothbrush of claim 1, wherein the bristles in the plurality of bristles are made of miswak, nylon, miswak extract infused nylon, or a combination thereof.

9. The toothbrush of claim 1, wherein the handle is curved from front to rear.

10. The toothbrush of claim 1, wherein the handle is wider at a bottom end thereof than at a top end where it couples with the head.

11. The toothbrush of claim 1, wherein the head has a substantially flat upper surface and an arc-shaped lower surface, with the bristles extending from the upper surface of the head.

12. The toothbrush of claim 1, wherein the head has a width that is greater than the width of the handle.

13. The toothbrush of claim 1, wherein an upper part of the handle is angled, and the plurality of bristles longitudinally align with the angle of the upper part of the handle.

14. The toothbrush of claim 1, wherein an upper part of the handle is angled, and the plurality of bristles are not longitudinally aligned with upper part of the handle.

15. The toothbrush of claim 1, wherein the handle has a width that is greater than a depth thereof.

16. The toothbrush of claim 1, wherein in use, the bristles are angled at an angle that along with the typical brushing angle provides an overall brushing angle of 45° relative to a side surface of a user's tooth.

17. A toothbrush comprising:
a T-shaped member having a handle forming the lower part of the T and a head forming the upper, transverse part of the T; and
a plurality of bristles extending outwardly from the head;
wherein the combination of the head, the bristles, and a typical toothbrush holding position of a user together provides a 45-degree angle relative to a side surface of a tooth for brushing.

18. The toothbrush of claim 17, wherein a normal toothbrush holding position of a user provides an angle ranging from about 10° to about 20° relative to a side surface of a user's tooth.

19. The toothbrush of claim 17, wherein the bristles of the toothbrush are angled at an upper end thereof at an angle of about 25° to about 35° relative to the side surface of a tooth of a user to provide a 45° brushing angle.
